(19) United States
(12) Patent Application Publication
(10) Pub. No.: US 2023/0253123 A1
(43) Pub. Date: Aug. 10, 2023

(54) SAFE PACKAGE HANDLING

(71) Applicant: INTERNATIONAL BUSINESS MACHINES CORPORATION, ARMONK, NY (US)

(72) Inventors: Paul R. Bastide, Ashland, MA (US); Jayanth Krishnan, Mahopac, NY (US); Piyush Madan, Boston, MA (US); Fang Lu, Billerica, MA (US)

(21) Appl. No.: 17/650,391

(22) Filed: Feb. 9, 2022

(51) Int. Cl. G16H 50/80; G16H 50/20; G06Q 10/08

(52) U.S. Cl. CPC G16H 50/80; G16H 50/20; G06Q 10/0832; G06Q 10/087

(57) ABSTRACT

A method, a structure, and a computer system for safe package handling. The exemplary embodiments may include identifying a shipping route of one or more packages, as well as determining a disease density and population health along the shipping route. The exemplary embodiments may further include determining whether the package is likely to transmit a disease contracted along the shipping route to a recipient of the package based on applying a model to the disease density and the population health. Based on determining that the package is likely to transmit a disease to the recipient, exemplary embodiments may include reducing disease transmission risk of the package.

### BACKGROUND

The exemplary embodiments relate generally to healthcare, and more particularly to safe package handling.

A common means for the transfer of goods is through the shipping of packages. While shipping may add transactional convenience for a sender and recipient, shipping packages may also presents a medium for the spread of a virus or bacteria. Moreover, the number of points of exchange during the shipping process may present additional opportunity for the spread of disease via a package.

### SUMMARY

The exemplary embodiments disclose a method, a structure, and a computer system for safe package handling. The exemplary embodiments may include identifying a shipping route of one or more packages, as well as determining a disease density and population health along the shipping route. The exemplary embodiments may further include determining whether the package is likely to transmit a disease contracted along the shipping route to a recipient of the package based on applying a model to the disease density and the population health. Based on determining that the package is likely to transmit a disease to the recipient, exemplary embodiments may include reducing disease transmission risk of the package.

### BRIEF DESCRIPTION OF THE SEVERAL VIEWS OF THE DRAWINGS

The following detailed description, given by way of example and not intended to limit the exemplary embodiments solely thereto, will best be appreciated in conjunction with the accompanying drawings, in which:

FIG. 1 depicts an exemplary schematic diagram of a safe package handling system 100, in accordance with the exemplary embodiments.

FIG. 2 depicts an exemplary flowchart 200 illustrating the analytics pipeline of a safe package handling program 132 of the safe package handling system 100, in accordance with the exemplary embodiments.

FIG. 3 depicts an exemplary block diagram depicting the hardware components of the safe package handling system 100 of FIG. 1, in accordance with the exemplary embodiments.

FIG. 4 depicts a cloud computing environment, in accordance with the exemplary embodiments.

FIG. 5 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

Detailed embodiments of the claimed structures and methods are disclosed herein; however, it can be understood that the disclosed embodiments are merely illustrative of the claimed structures and methods that may be embodied in various forms. The exemplary embodiments are only illustrative and may, however, be embodied in many different forms and should not be construed as limited to the exemplary embodiments set forth herein. Rather, these exemplary embodiments are provided so that this disclosure will be thorough and complete, and will fully convey the scope to be covered by the exemplary embodiments to those skilled in the art. In the description, details of well-known features and techniques may be omitted to avoid unnecessarily obscuring the presented embodiments.

References in the specification to “one embodiment”, “an embodiment”, “an exemplary embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is focused on the distinctive features or elements according to the various exemplary embodiments.

A common means for the transfer of goods is through the shipping of packages. While shipping may add transactional convenience for a sender and recipient, shipping packages may also presents a medium for the spread of a virus or bacteria. Moreover, the number of points of exchange during the shipping process may present additional opportunity for the spread of disease via a package.

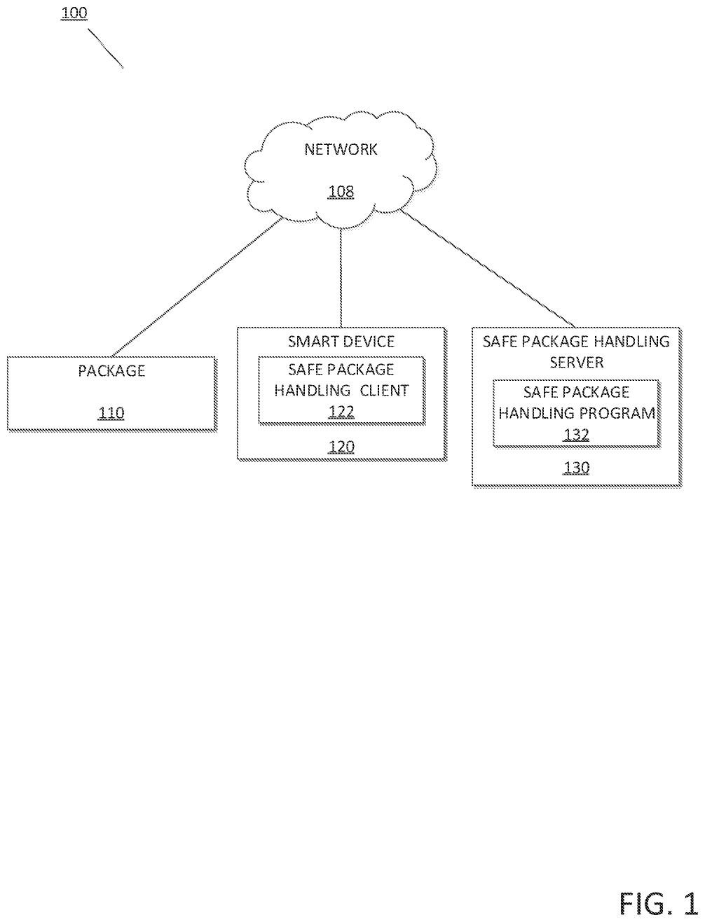

FIG. 1 depicts the safe package handling system 100, in accordance with exemplary embodiments. According to the exemplary embodiments, the safe package handling system 100 may include a package 110, a smart device 120, and a safe package handling server 130, which all may be interconnected via a network 108. While programming and data of the exemplary embodiments may be stored and accessed remotely across several servers via the network 108, programming and data of the exemplary embodiments may alternatively or additionally be stored locally on as few as one physical computing device or amongst other computing devices than those depicted. The operations of the safe package handling system 100 are described in greater detail herein.

In the exemplary embodiments, the network 108 may be a communication channel capable of transferring data between connected devices. In the exemplary embodiments, the network 108 may be the Internet, representing a worldwide collection of networks and gateways to support communications between devices connected to the Internet. Moreover, the network 108 may utilize various types of connections such as wired, wireless, fiber optic, etc., which may be implemented as an intranet network, a local area network (LAN), a wide area network (WAN), a combination thereof, etc. In further embodiments, the network 108 may be a Bluetooth network, a Wi-Fi network, a combination thereof, etc. The network 108 may operate in frequencies including 2.4 gHz and 5 gHz internet, near-field communication, etc. In yet further embodiments, the network 108 may be a telecommunications network used to facilitate telephone calls between two or more parties comprising a landline network, a wireless network, a closed network, a satellite network, a combination thereof, etc. In general, the network 108 may represent any combination of connections and protocols that will support communications between connected devices.

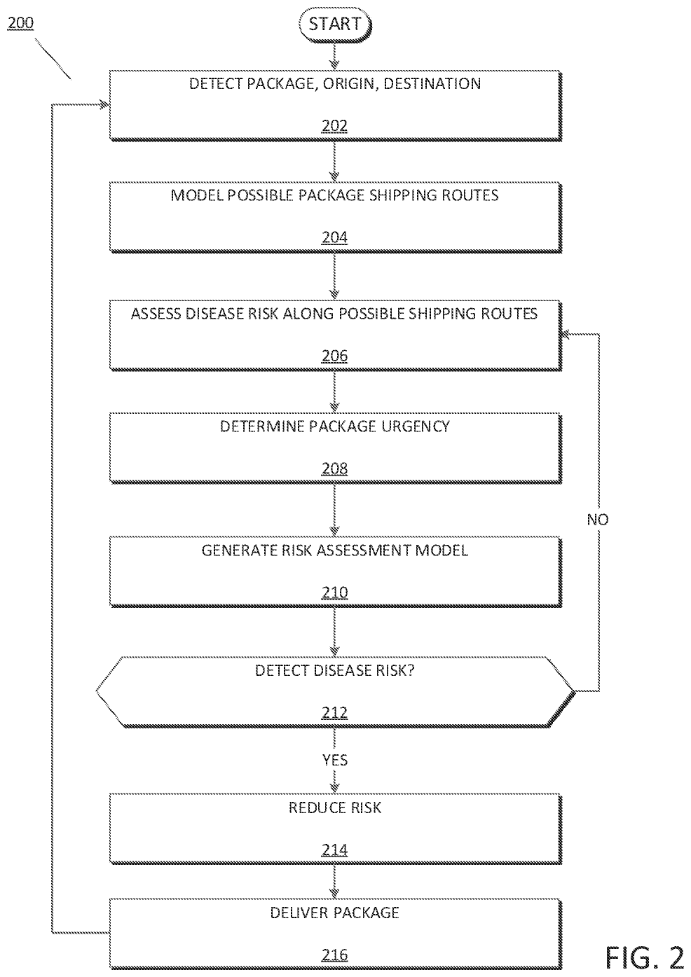

In exemplary embodiments, the package 110 may be a container for shipping goods, and may therefore be an envelope, box, crate, etc. The package 110 may be made of various materials, including paper, cardboard, wood, metal, fiberglass, composites, etc. In embodiments, the package 110 may be shipped from an origin to a destination, and may stop at one or more exchange locations, all of which collectively referred to as a shipping route. The shipping route may include multiple shipping carriers and multiple shipping means, such as ground, rail, and air. Throughout shipping, the package 110 may be subject to disease exposure from a sender of the package 110 and personnel of the shipping carrier, thus exposing to disease a recipient of the package 110. In embodiments, it is possible for the package 110 to carry and transfer such diseases without additional precaution, such as package redirection, repackaging, isolation, etc. The package 110 and those additional precautions are described in greater detail with respect to FIG. 2.

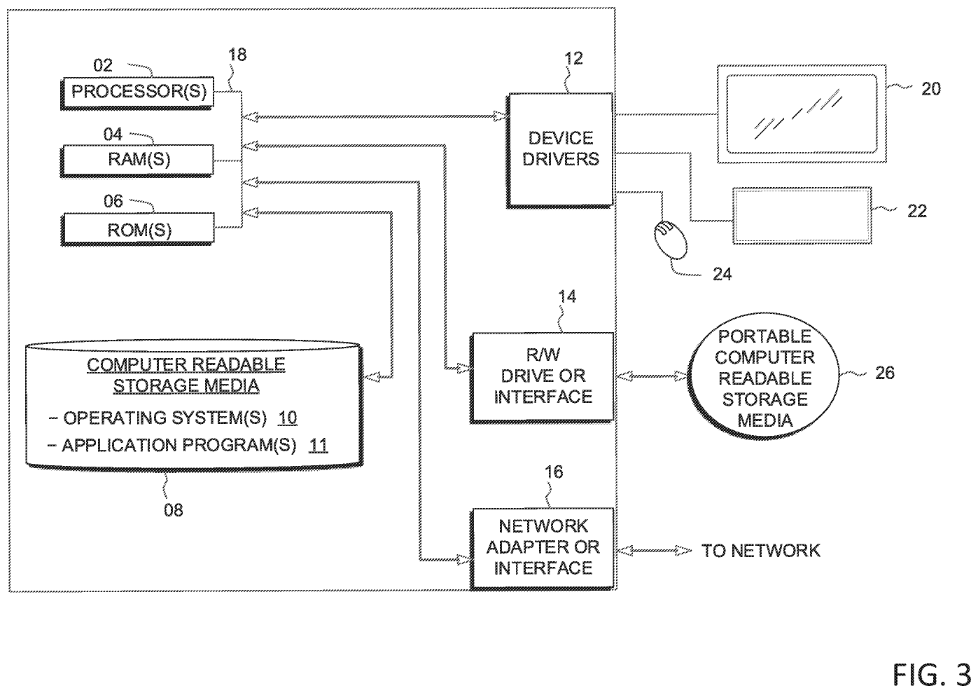

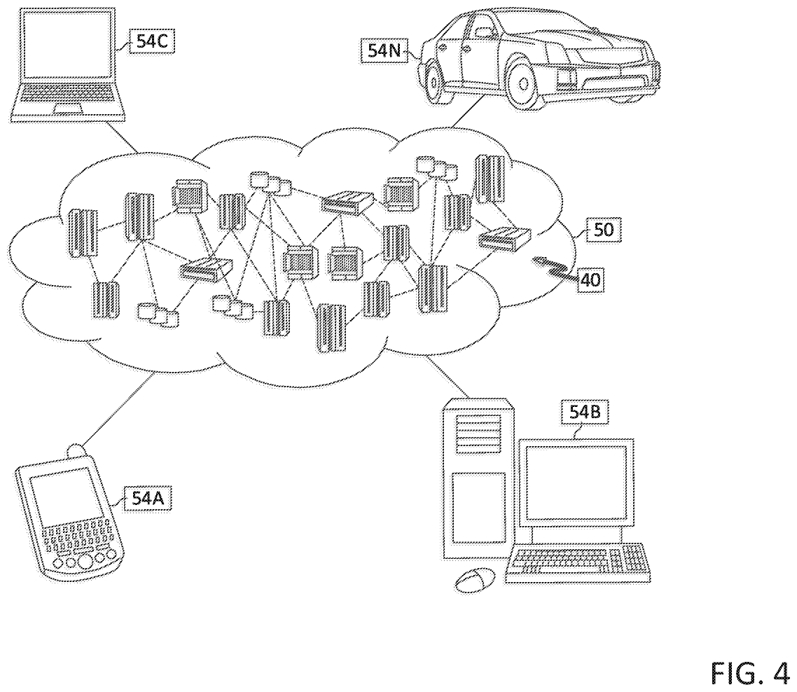

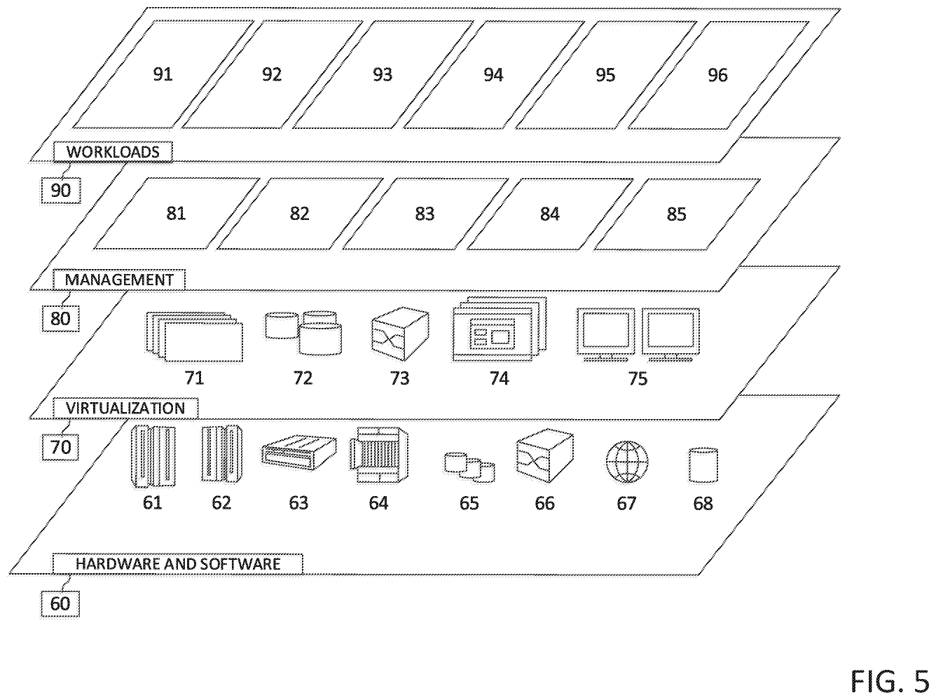

In exemplary embodiments, the smart device 120 includes a safe package handling client 122, and may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the smart device 120 is shown as a single device, in other embodiments, the smart device 120 may be comprised of a cluster or plurality of computing devices, in a modular manner, etc., working together or working independently. The smart device 120 is described in greater detail as a hardware implementation with reference to FIG. 3, as part of a cloud implementation with reference to FIG. 4, and/or as utilizing functional abstraction layers for processing with reference to FIG. 5.

The safe package handling client 122 may act as a client in a client-server relationship, e.g., with the safe package handling server 130, and may be a software and/or hardware application capable of communicating with and providing a user interface for a user to interact with the safe package handling server 130 and other computing devices via the network 108. Moreover, the safe package handling client 122 may be further capable of transferring data from the smart device 120 to and from other devices via the network 108. In embodiments, the safe package handling client 122 may utilize various wired and wireless connection protocols for data transmission and exchange, including Bluetooth, 2.4 gHz and 5 gHz internet, near-field communication (NFC), etc. The safe package handling client 122 may be capable of detecting when the package 110 is shipped, as well as a shipping carrier, origin, and destination of the package 110. The safe package handling client 122 may be further capable of receiving user input detailing health conditions of a user or household members thereof. The safe package handling client 122 is described in greater detail with respect to FIG. 2-5.

In exemplary embodiments, the safe package handling server 130 includes a safe package handling program 132, and may act as a server in a client-server relationship with the safe package handling client 122. The safe package handling server 130 may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the safe package handling server 130 is shown as a single device, in other embodiments, the safe package handling server 130 may be comprised of a cluster or plurality of computing devices, in a modular manner, etc., working together or working independently. The safe package handling server 130 is described in greater detail as a hardware implementation with reference to FIG. 3, as part of a cloud implementation with reference to FIG. 4, and/or as utilizing functional abstraction layers for processing with reference to FIG. 5.

The safe package handling program 132 may be a software and/or hardware program that may detect the shipping of a package, as well as an origin and a destination thereof. The safe package handling program 132 may additionally model possible package shipping routes and assess disease risk along the possible shipping routes. The safe package handling program 132 may further determine package urgency and generate a risk model based on the assessed risk. The safe package handling program 132 may additionally determine whether a risk of disease is detected along the shipping route and, based thereon, reduce risk before delivering the package. The safe package handling program 132 is described in greater detail with reference to FIG. 2-5.

FIG. 2 depicts an exemplary flowchart 200 illustrating the operations of the safe package handling program 132 of the safe package handling system 100, in accordance with the exemplary embodiments.

The safe package handling program 132 may detect the shipping of a package, as well as an origin and destination thereof (step 202). In embodiments, the safe package handling program 132 may detect the shipping of the package 110 via several means. In an embodiment, the safe package handling program 132 may detect the package 110 has been shipped via user input, e.g., manual entry of a tracking number or scanning of a tracking barcode. In other embodiments, the safe package handling program 132 may detect that the package 110 has shipped via the safe package handling client 122 intercepting information regarding the shipment of package 110 from the smart device 120, e.g., by extracting shipment data based on the recognition of key words, tracking numbers, etc. In other embodiments, the safe package handling program 132 may detect the shipping of the package 110 via integration with a shipping carrier or scheduling service that share shipment information with the safe package handling program 132. In such embodiments, the safe package handling program 132 may work with the shipping carrier or scheduling service in order to plan a safest shipping route for the package 110. Alternatively, the safe package handling program 132 may be configured to alert the shipping carrier or scheduling service of shipments of the package 110 that are high risk to transfer disease to a recipient.

The shipping information may include an originating location (origin) from which the package is shipped, a destined location (destination), a shipping carrier, a shipping class of the package 110 (e.g., first class, overnight, etc.) indicative of a timeframe in which the package is to be delivered, whether a signature is required for delivery, and details regarding an exterior of the package 110 itself, for example dimensions, exterior material, etc. In embodiments, and with consent from all required parties, the safe package handling program 132 may further extract information regarding a contents of the package 110, or classify the contents of the package 110 for urgency consideration.

In embodiments, the safe package handling program 132 may utilize general and objective population data to assess risk of the package 110 contracting a disease at exchange locations along the shipping route, as well as the recipient contracting such diseases from the package 110. However, should users consent, the safe package handling program 132 may receive subjective health information of the sender and/or recipient of the package 110, as well as households thereof. The health information may include demographics such as age and location, as well as information indicative of whether the sender or recipient (or household member thereof) is susceptible to (or has) a disease, is part of any particular patient cohort, has pre-existing conditions, etc. Whether objective and/or subjective, the health information may inform determining a risk that a recipient of the package 110 will contract a disease therefrom. In particular, the safe package handling program 132 may assess a chance that the sender (as well as any carrier) contract a disease and pass it to the package 110 (e.g., based on geography disease information), as well as a chance that the recipient contract the disease from the package 110. Such operations are described in greater detail forthcoming.

In order to better illustrate the operations of the safe package handling program 132, reference is now made to an illustrative example wherein the safe package handling program 132 detects a sender scheduling for the shipping of a cardboard box containing medical supplies from location A to location D by carrier X.

The safe package handling program 132 may model possible shipping routes of the package (step 204). In embodiments, the safe package handling program 132 may identify one or more shipping routes, including one or more exchanges between carriers, that the package 110 may follow as it travels from the origin to the destination. In embodiments where not all information is known (e.g., only an origin, destination, and carrier are extracted), the safe package handling program 132 may identify a list of all routes run by the carrier between the origin to the destination, including ground (vehicle, truck, train, etc.) and air (plane, etc.) freight. The safe package handling program 132 may additionally filter routes based on the timeframe of the shipping class in which the package is to be delivered. The safe package handling program 132 may then select a most likely shipping route of the one or more potential shipping routes that the package 110 may be shipped. In some embodiments, the safe package handling program 132 may be configured to select two or more most likely shipping routes and run similar analysis described herein on each potential route. In embodiments described above where the safe package handling program 132 is integrated with the shipping carrier or scheduling service that shares shipping information, the safe package handling program 132 may be capable of extracting the planned shipping route of the package 110.

The possible shipping route information may further detail the means of transportation of the package 110, as well as points of contact at any exchanges when the package 110 leaves one carrier for the next. The exchanges may be, e.g., moving the package 110 from one vehicle to another, into temporary storage, etc., and the points of contact at exchanges may involve human- or machine-based exchanges. Notably, at each exchange, the human/machine potentially expose the package 110 directly or indirectly to diseases within the exchange location area. In embodiments, the safe package handling program 132 may extract exchange and points of contact information from the carrier, e.g., information indicative of human/machine staffing or operation. The point of contact information may further include and extent and a duration of the points of contact, e.g., as determined by timestamps on shipping data, video analysis, etc. Moreover, such point of contact information may additionally include protocol implemented to reduce disease transmission, i.e., transmission-based precautions, such as the use of antibacterial, masks, gloves, ventilation filtration, UV light, etc.

As previously mentioned, the safe package handling program 132 may operate using objective health information of populations surrounding exchange locations along the shipping route. Again, however, should shipping carrier personnel consent, the possible shipping route information may further include the health information of shipping personnel in contact with the package 110, e.g., those working at arrival and departure times of the package 110 at the origin, destined, and exchange locations. Similar to the health information optionally volunteered by the sender and recipient, the health information consensually provided by the carrier personnel may include demographics such as age and location, as well as information indicative of whether the carrier personnel (or household member thereof) is susceptible to (or has) a disease, is part of any particular patient cohort, has pre-existing conditions, etc.

Furthering the illustrative example introduced above, the safe package handling program 132 may identify shipping routes 1 as a most likely shipping route of the package 110 by carrier X (alternatively, shipping route 1 may be positively indicated by the carrier X), which has exchange locations B and C between originating location A and destined location D.

The safe package handling program 132 may assess disease risk along the shipping route (step 206). In embodiments, the safe package handling program 132 may assess disease risk along the shipping route objectively based on population data at the origin (e.g., pickup), any exchange locations, and the destination (e.g., when out for delivery). When applicable, the safe package handling program 132 may further assess disease risk subjectively based on volunteered health information of the users, namely that of the sender when considering the originating location, that of the shipping carrier personnel at any of the exchange locations, and that of the recipient at the destined location. As noted previously, however, the provision of subjective health information is strictly voluntary and disease transmission risk may be computed based on objective disease and health information along the shipping route.

The safe package handling program 132 may assess disease risk based on health information that details the diseases as well as severity and pervasiveness thereof in the geographical areas of the shipping route. The disease risk information may take into consideration the objective health of the population within the area under consideration, as well as that volunteered by a user, which may further enhance the analysis. The health information may include data relevant to disease susceptibility, such as age, pre-existing conditions, etc., which may be aggregated in the objective data. The disease information may include disease type, transmission medium (airborne, droplet, object, food and water, animal-to-person contact, animal reservoirs, insect bites, environmental reservoirs, etc.), surface life, disease prevention/disinfectant, disease eradication, etc. In addition, the disease severity and pervasiveness data may detail disease concentration, disease outbreaks, disease contagiousness, disease symptoms, etc. The safe handling program 132 may retrieve the disease and disease pervasiveness information from various public and/or private resources, such as hospital records, medical databases, educational institutions, government agencies, etc. The disease and disease pervasiveness information may further identify disease hotspots, localized outbreaks, susceptible cohorts of people, susceptible surfaces for extended surface life/transfer, ideal disease transmission conditions, etc.

With reference again to the formerly introduced example, the safe package handling program 132 may assess one or more diseases and general population health at locations A, B, C, and D, identifying a high density of infectious disease breakout at exchange location B. Should the sender, carrier personnel, and recipient volunteer personal health information, the safe package handling program 132 may additionally retrieve the health of the sender at the origin, the health of the carrier personnel at the exchange locations, and the health of the recipient at the destination.

The safe package handling program 132 may determine an urgency of the delivery of the package 110 (step 208). In embodiments, the urgency to deliver the package 110 may inform a decision (if deemed necessary) regarding how to reduce risk of disease transmission to the recipient while still delivering the package 110 within delivery timeframes. The safe package handling program 132 may accordingly prioritize the shipping of the package 110 when containing high priority contents over those containing contents having no urgency. In embodiments, the safe package handling program 132 may prioritize the package 110 based on contents, e.g., a category or intended use of the package 110 (e.g., medical, business, etc.), where contents having a higher prioritized intended use may be selectively chosen for avoidance of time-consuming risk reduction practices, e.g., redirecting or repackaging vs. hot/cold storage for a cooldown period. Such prioritization may be particularly advantageous in situations where shipping is overwhelmed/delayed and/or risk reducing techniques are limited, thus ensuring delivery of essential shipments. The prioritized categories may include contents relating to, e.g., medical, safety, emergency, etc., supplies.

In the aforementioned example, the safe package handling program 132 determines that the package 110 is high urgency due to containing medical supplies, and therefore delivery cannot be delayed.

The safe package handling program 132 may generate a risk assessment model (step 210). In exemplary embodiments, the safe package handling program 132 may generate a risk assessment model that may be applied to determine a risk of the package 110 passing to the recipient a disease. In particular, the safe package handling program 132 may determine that risk based on a chance of the package 110 contracting the disease along the shipping route (i.e., at any of the origin, destination, and exchanges therebetween), as well as a chance that the recipient contract the disease from the package 110. The risk assessment model may be based on machine learning, e.g., a neural network, and receive input features that may include the objective population or subjective (i.e., sender, carrier personnel, and recipient, as well as households thereof) health conditions, as well as the diseases along the shipping route (including a severity and a pervasiveness thereof). Additional features may include an exterior of the package 110, the extent and duration of points of contact at the exchanges, transmission-based precautions at points of contact, content of the package 110, whether a signature is required at delivery, weather, temperature, atmospheric conditions, etc. Overall, the model may be fed any features relating to disease transmission.

The risk assessment model may be trained with training data that details historic values of the features when shipping-based disease transmission occurs and does not occur. The training data may, e.g., be labelled by a human in supervised learning or deduced by the safe package handling program 132 in unsupervised learning. During training, the risk assessment model may be trained to recognize feature values indicative of increased disease transmission, and thus increased risk to the recipient. The risk assessment model may be trained until high disease transmission risk is accurately recognized. After the risk assessment model is trained sufficiently, e.g., reaches a threshold level of accuracy, the model may be configured to receive real-time feature values for comparison to the modelled values, allowing for the output of a risk value indicative of overall chances of recipient disease contraction.

Returning to the aforementioned example, the safe package handling program 132 generates the risk assessment model using training data that details historic feature values when package-based disease transmission occurs and does not occur.

The safe package handling program 132 may determine whether a disease risk is detected (decision 212). In embodiments, the safe package handling program 132 may determine whether a risk is detected based on applying the risk assessment model to the objective or subjective health conditions and the disease information. In embodiments, the risk assessment model may output a risk assessment value that is indicative of disease transmission risk. The safe package handling program 132 may then compare the risk assessment value to a threshold indicative of a need to take risk reduction measures. If the risk assessment value exceeds the threshold, the safe package handling program 132 detects a disease risk.

With reference to the previously introduced example, the safe package handling program 132 applies the risk assessment model to objective health information (and optionally subjective health information of the sender, recipient, and personnel) and the disease information at locations A, B, C, and D. The risk assessment model outputs risk assessment values for each of locations A, B, C, and D that are compared to a threshold in order to determine that there is a high risk of disease transmission at exchange location B.

Based on determining that a disease risk is not detected (decision 212, “NO” branch), the safe package handling program 132 may continue to assess disease risk along the shipping route until the package 110 has been delivered to the recipient. The safe package handling program 132 may, e.g., reassess risk at each exchange, at periodic intervals, upon specific triggers, etc. The specific triggers may include, e.g., a change in shipping route, a change in carrier personnel, a change in the contents or external material of the package 110, a change in disease information, a change in health information, etc.

Alternatively, based on determining that a disease risk is detected (decision 212, “YES” branch), the safe package handling program 132 may reduce risk (step 214). In exemplary embodiments, the safe package handling program 132 may reduce risk using techniques such as redirecting the package 110 through a different exchange location, repackaging the product at any point during the shipment, delaying delivery to the recipient until a minimum hot/cold storage time has elapsed (e.g., based on disease), treating the product (e.g., with an antifungal, antiviral, or disinfecting agent), etc. In embodiments, the safe package handling program 132 may select a means for reducing disease transmission risk based on the disease, the exterior of the package 110, the urgency, the means available at/to the particular exchange, etc. In embodiments, the package 110 having prioritized urgency may be afforded the risk reduction means having a shortest duration such that an intended delivery window may be maintained.

Depending on the availability of alternative shipping routes, short duration risk reduction means may include redirection of the package 110 around the geography in which a disease risk is detected. In such embodiments, the safe package handling program 132 may identify alternative shipping routes and perform a similar disease risk analysis as applied above, identifying a suitable alternative and an added shipping duration before determining whether the delivery window is maintained based thereon. In addition, and based on potential disease exposure, the safe package handling program 132 may recommend the short duration risk reduction means of safe repackaging or disinfecting of the package 110. The repackaging may be with a material more suitable (e.g., less susceptible to disease) or disposable (e.g., a sealed bag) for travel through an area having a high disease concentration, or with a new and/or sanitized packaging after travel through a high risk area. The sanitization may include a washing, disinfecting, antibacterial, antimicrobial, UV disinfecting, cold/hot/vacuum exposure, etc. Alternatively, for the package 110 having less urgency and again based on the potential disease exposure, the safe package handling program 132 may recommend hot/cold storage for a particular duration. Overall, the safe package handling program 132 may recommend any means for reducing risk that are available to it.

The safe package handling program 132 may implement the risk reduction means. In embodiments, risk reduction means may require the safe package handling program 132 modify a shipping label associated with the package 110 such that the package 110 is redirected to an alternative exchange location, or transferred to a repackaging or disinfecting station. In embodiments having warehouses with autonomous machinery, the safe package handling program 132 may be configured to communicate such risk reduction means to the machinery (e.g., relabelling, redirecting, sanitizing, repackaging, hot/cold storage, etc.) for autonomous performance, thereby avoiding contact of the package 110 with carrier personnel. These autonomous operations may be twofold in that diseases carried by the package 110 may not be transferred to carrier personnel, and vice versa. The risk reduction means may then be carried out before the package 110 is reintroduced within the shipping route.

In furthering the example introduced above, the safe package handling program 132 determines that redirecting the package 110 to location B′ instead of location B will reduce disease transmission risk while maintaining the appropriate delivery window of the medical supplies. The sage package handling program 132 relabels the package 110 to reflect the change in shipping route 1.

The safe package handling program 132 may deliver the package (step 216). In exemplary embodiments, the safe package handling program 132 may deliver the package after having completed the risk reduction means. The safe package handling program 132 may ensure the package 110 no longer presents a risk of disease contraction to the recipient by, e.g., reiterating the risk reduction means or, if applicable, testing the package 110 for disease. Once the safe package handling program 132 ensures that the risk of disease contraction is reduced, the package 110 may be delivered. In embodiments, the safe package handling program 132 may deliver the package 110 via machine such that human contact is further avoided following such risk reduction measures. The package 110 may be delivered via, e.g., a robot or drone. The machine may undergo risk reduction means (e.g., sanitization), or be comprised of materials less susceptible to disease in order to further lessen the chance of passing a disease to the recipient.

Concluding the aforementioned example, the safe package handling program 132 delivers the package to the recipient at the destined location after redirection to B′.

FIG. 3 depicts a block diagram of devices used within safe package handling system 100 of FIG. 1, in accordance with the exemplary embodiments. It should be appreciated that FIG. 5 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a RAY drive or interface 14 to read from and write to one or more portable computer readable storage media 26. Application programs 11 on said devices may be stored on one or more of the portable computer readable storage media 26, read via the respective RAY drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, R/W drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and safe package handling processing 96.

The exemplary embodiments may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-implemented method for safe package handling, the method comprising:
identifying a shipping route of one or more packages;
determining a disease density and population health along the shipping route;
determining whether the package is likely to transmit a disease contracted along the shipping route to a recipient of the package based on applying a model to the disease density and the population health; and
based on determining that the package is likely to transmit a disease to the recipient, reducing disease transmission risk of the package.

2. The computer-implemented method of claim 1, wherein reducing disease transmission risk of the package further comprises at least one of redirecting the package, repackaging the package, sanitizing the package, and temporarily storing the package.

3. The computer-implemented method of claim 1, wherein the population health includes health information corresponding to at least one of a sender, the recipient, and shipping personnel of the package.

4. The computer-implemented method of claim 1, wherein the model correlates a chance of the package contracting a disease with the disease density and the population health at an origin, a destination, and one or more shipping exchanges therebetween of the package.

5. The computer-implemented method of claim 1, wherein the model further correlates a chance of the package transmitting the disease to the recipient with the chance of the package contracting the disease.

6. The computer-implemented method of claim 1, wherein determining whether the package is likely to transmit a disease to the recipient is further based on an external material of the package.

7. The computer-implemented method of claim 1, further comprising:
delivering the package.

8. A computer program product for safe package handling, the computer program product comprising:
one or more non-transitory computer-readable storage media and program instructions stored on the one or more non-transitory computer-readable storage media capable of performing a method, the method comprising:
identifying a shipping route of one or more packages;
determining a disease density and population health along the shipping route;
determining whether the package is likely to transmit a disease contracted along the shipping route to a recipient of the package based on applying a model to the disease density and the population health; and
based on determining that the package is likely to transmit a disease to the recipient, reducing disease transmission risk of the package.

9. The computer program product of claim 8, wherein reducing disease transmission risk of the package further comprises at least one of redirecting the package, repackaging the package, sanitizing the package, and temporarily storing the package.

10. The computer program product of claim 8, wherein the population health includes health information corresponding to at least one of a sender, the recipient, and shipping personnel of the package.

11. The computer program product of claim 8, wherein the model correlates a chance of the package contracting a disease with the disease density and the population health at an origin, a destination, and one or more shipping exchanges therebetween of the package.

12. The computer program product of claim 8, wherein the model further correlates a chance of the package transmitting the disease to the recipient with the chance of the package contracting the disease.

13. The computer program product of claim 8, wherein determining whether the package is likely to transmit a disease to the recipient is further based on an external material of the package.

14. The computer program product of claim 8, further comprising:
delivering the package.

15. A computer system for safe package handling, the system comprising:
one or more computer processors, one or more computer-readable storage media, and program instructions stored on the one or more of the computer-readable storage media for execution by at least one of the one or more processors capable of performing a method, the method comprising:
identifying a shipping route of one or more packages;
determining a disease density and population health along the shipping route;
determining whether the package is likely to transmit a disease contracted along the shipping route to a recipient of the package based on applying a model to the disease density and the population health; and
based on determining that the package is likely to transmit a disease to the recipient, reducing disease transmission risk of the package.

16. The computer system of claim 15, wherein reducing disease transmission risk of the package further comprises at least one of redirecting the package, repackaging the package, sanitizing the package, and temporarily storing the package.

17. The computer system of claim 15, wherein the population health includes health information corresponding to at least one of a sender, the recipient, and shipping personnel of the package.

18. The computer system of claim 15, wherein the model correlates a chance of the package contracting a disease with the disease density and the population health at an origin, a destination, and one or more shipping exchanges therebetween of the package.

19. The computer system of claim 15, wherein the model further correlates a chance of the package transmitting the disease to the recipient with the chance of the package contracting the disease.

20. The computer system of claim 15, wherein determining whether the package is likely to transmit a disease to the recipient is further based on an external material of the package.
